(19) United States
(12) Patent Application Publication
(10) Pub. No.: US 2022/0074000 A1
(43) Pub. Date: Mar. 10, 2022

(54) PATHOGEN TEST SYSTEMS AND METHODS

(71) Applicants: Christopher Wong, San jose, CA (US); Lyle Probst, Burlingame, CA (US); Bao Tran, Saratoga, CA (US); Matthew Grimm, Burlingame, CA (US); Jason Lohe, Burlingame, CA (US); Barney Lee, sanjose, CA (US); Michael Massing, san jose, CA (US)

(72) Inventors: Christopher Wong, San jose, CA (US); Lyle Probst, Burlingame, CA (US); Bao Tran, Saratoga, CA (US); Matthew Grimm, Burlingame, CA (US); Jason Lohe, Burlingame, CA (US); Barney Lee, sanjose, CA (US); Michael Massing, san jose, CA (US)

(21) Appl. No.: 17/017,643

(22) Filed: Sep. 10, 2020

(51) Int. Cl. C12Q 1/6888; G06F 21/32; G06F 21/33; G06N 20/00; H04W 4/029

(52) U.S. Cl. CPC C12Q 1/6888; G06F 21/32; H04W 4/029; G06N 20/00; G06F 21/33

(57) ABSTRACT

A method to test a person includes capturing genetic material from a person being tested; performing a RNA and or DNA or genetic test on the captured material for a predetermined pathogen; if the test shows an absence of the predetermined pathogen, certifying the person with a pathogen free status; and providing a certificate during travel or entrance to a facility as proof of pathogen free status. The method can optionally use a mobile testing lab that includes a mobile test platform including a sealed environment that is easily disinfected; a plurality of storage spaces to store virus genetic testing equipment, diagnostic computer, support equipment, a fume hood with an internal filter and no outside venting; and a power generator.

This application claims priority to Provisional Application Ser. 63/017,532 filed Apr. 29, 2020, the content of which is incorporated by reference.

The present invention relates to rapid testing for pathogens.

Many diseases can spread quickly in the human population, some of which are caused by viruses. Coronaviruses are a large group of viruses that cause diseases in animals and humans. They often circulate among animals and can sometimes evolve and infect people. A novel coronavirus (SARS-CoV-2) that causes the disease Coronavirus Disease 2019 (COVID-19) emerged in a seafood and poultry market in Wuhan, China in December 2019. Cases have been detected in most countries worldwide, and on Mar. 11, 2020, the World Health Organization characterized the outbreak as a pandemic. Human-to-human transmission occurs through close contact.

There is evidence that the novel coronavirus and other communicable diseases can be spread before an individual develops symptoms. This poses a problem because people who do not know they are infected may continue to go to work, school, and other public places. People who are sick and have symptoms are more likely to stay home, which means fewer opportunities for the virus to spread from one person to another. When asymptomatic transmission occurs, infection control experts and public health officials may need to take additional measures, such as social distancing, isolating patients, or using quarantines.

COVID-19 is affecting normal population and crippling industry, and it is spreading at an accelerated rate due to lack of testing and not adhering to CDC regulations. After the initial wave of infection which was controlled through strict quarantine measures, the pandemic's resurgence, including in places where it had been contained, makes it clear that for the foreseeable future the risk of COVID-19 can't be eliminated, only managed. One of the keys to reopening the economy is having enough tests to diagnose coronavirus infections, with the goal being to quickly identify new cases, isolate them, and track down others who may have been exposed.

### SUMMARY OF THE INVENTION

In a first aspect, a process to provide a mobile paper or digital certificate/passport for a person includes capturing genetic virus or bacteria material from a person being tested. The system then performs an RNA and or DNA Analysis on the captured material. If the test shows an absence of Pathogen, the method certifies that the person “Passed Health Testing” in a Digital Certificate and otherwise marks the Digital Certificate with “Failed Health Testing.” The Digital Certificate can be a mobile app, an icon such as a 2D or 3D bar code, or an entry on a blockchain that can be accessed by third parties. The Digital Certificates can be scanned via wireless scanners by a guard or an access station and based on the reports contained therein, can act as an electronic passport to enable a person to have clearance for travel, work, or study, among others.

In a second aspect, the system includes RNA and DNA testing and or sequencing equipment with computer code to:
Capture COVID Genetic Material from Person Being Tested
Perform RNA and DNA Analysis on the Captured Material
If Test Shows Absence of COVID viruses, Certify Person as “Passed COVID Testing” in a Digital Certificate
Otherwise Mark the Digital Certificate as “Failed COVID Testing”

In a third aspect, the system includes RNA and DNA testing and or sequencing equipment with computer code to:
Capture Genetic Virus or Bacteria Material from Person Being Tested
Perform RNA and DNA Analysis on the Captured Material
If Test Shows Absence of SARS Pathogen, Certify Person as “Passed SARS Testing” in a Digital Certificate
Otherwise Mark the Digital Certificate as “Failed SARS Testing”

In a fourth aspect, the system includes RNA and DNA testing and or sequencing equipment with computer code to:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a home RNA and or DNA tester
If Test Shows Absence of COVID Pathogen, Certify Person as “Passed Health Tests” in a Digital Certificate
Otherwise Mark the Digital Certificate as “Failed Health Tests”

In a fifth aspect, the system includes RNA and DNA testing and or sequencing equipment with computer code to:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a home RNA and or DNA tester
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities

In a sixth aspect, the system includes RNA and DNA testing and or sequencing equipment with computer code to:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a package with a preselected address for an approved testing lab
When received at testing lab, an AI system determines likelihood that person has been exposed to COVID virus
If the person is unlikely to be COVID positive, testing lab runs pooled testing to speed up test capacity
Otherwise testing lab individually test the person's specimen
If Test Shows Absence of COVID Pathogen, Certify Person as “Passed Health Tests” in a Digital Certificate
Otherwise Mark the Digital Certificate as “Failed Health Tests”

In a seventh aspect, the system includes RNA and DNA testing and or sequencing equipment with computer code to:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a package with a preselected address for an approved testing lab
When received at testing lab, an AI system determines likelihood that person has been exposed to COVID virus
If the person is unlikely to be COVID positive, testing lab runs pooled testing to speed up test capacity
Otherwise testing lab individually test the person's specimen
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities

In an eighth aspect, the system includes RNA and DNA testing and or sequencing equipment with computer code to:
Confirm identity of person using biometrics such as eyes, face, and fingerprint(s)
Once confirmed, ask person to insert test swabs at nose/ear/throat or spits saliva and collect personal test specimens and bring to a mobile testing lab or truck with genetic test equipment
AI system determines likelihood that person has been exposed to COVID virus
If the person is unlikely to be COVID positive, mobile testing lab runs pooled testing for the employees in one or more batches of specimens
Otherwise testing lab individually test the person's specimen
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities

In a ninth aspect, the system includes RNA and DNA testing and or sequencing equipment with computer code to:
Confirm identity of person using biometrics such as eyes, face, and fingerprint(s)
Once confirmed, ask person to insert test swabs at nose/ear/throat or spits saliva and collect personal test specimens and bring to a mobile testing lab or truck with genetic test equipment
AI system determines likelihood that person has been exposed to COVID virus
If the person is unlikely to be COVID positive, mobile testing lab runs pooled testing for the employees in one or more batches of specimens
Otherwise testing lab individually test the person's specimen
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities, send person to receive COVID treatment. Such treatment can include:
Stay home except to get medical care.
Monitor your symptoms carefully. If your symptoms get worse, call the healthcare provider immediately.
Get rest and stay hydrated. Take over-the-counter medicines, such as acetaminophen, to help feel better.
If a medical appointment, notify your healthcare provider ahead of time that you have or may have COVID-19.
Stay in a specific room and away from other people in your home. If possible, use a separate bathroom. If must be around others, wear a facemask.

Treatments can include Remdesivir, an antiviral that is given by intravenous (IV) infusion in the hospital. This is a brand-new drug that has not been approved by the FDA for use on the market yet, and is being tested in carefully controlled environments. It was previously shown to have some effect against SARS, MERS, and Ebola in cell and animal models. In a recent in vitro study (studies done in a petri dish or test tube rather than in animals or humans), Remdesivir prevented human cells from being infected with SARS-CoV-2 (the virus that causes COVID-19). Another treatment is Dexamethasone which is a common corticosteroid (steroid) medication that has been used for many years to treat various health conditions, such as autoimmune conditions and allergic reactions. RECOVERY, a randomized clinical trial in the UK, is studying many medications, including dexamethasone, to see if any are effective against COVID-19. Hydroxychloroquine and chloroquine are two medications that have been used for many decades to treat malaria and autoimmune conditions like rheumatoid arthritis and lupus. Azithromycin (informally known as a Z-pak) is an antibiotic commonly used to treat bacterial infections such as bronchitis and pneumonia. It has been shown to have some in vitro activity against viruses like influenza A and Zika, but did not work against the coronavirus that causes MERS. Convalescent plasma can be used to treat people with COVID-19. Plasma is the liquid part of blood that carries blood cells. Convalescent plasma is collected from people who have recovered from COVID-19. It is then transfused into someone with an active coronavirus infection. It is thought that antibodies found in the convalescent plasma can help fight the coronavirus infection.

In yet another aspect, a mobile testing lab includes a mobile test platform including a sealed environment that is easily disinfected; a plurality of storage spaces to store virus genetic testing equipment, diagnostic computer, support equipment, a Bio Safety Cabinet with an internal filter and no outside venting.

In implementations, the user can get vaccinated in the mobile testing lab. The vaccine can be built with mRNA, a piece of genetic code containing the instructions for the coronavirus' spike protein. The vaccine shuttles the mRNA into cells, which “read” those instructions and churn out the protein. Other vaccines can attach the gene for the spike protein to another, harmless virus that ferries it into cells. There, it gets expressed into the spike, allowing the immune system to start work. Another embodiment work on the spike proteins, which give SARS-CoV-2 the crown-like appearance that's characteristic of coronaviruses, attach to receptors on people's cells, allowing the virus to enter and replicate. By blocking spike proteins, then, vaccines could prevent infection. The spikes are also the “immunodominant” piece of the virus, meaning they elicit the strongest immune response.

In other implementations, a trailer for COVID-19 testing includes one or more of the following:

1. Self-contained bio safety cabinet, no outside venting with HEPA filtration

2. qPCR Equipment—Minimum Throughput/trailer/hour 92 (greater throughput can be achieved with medium or hi-capacity units). A plurality of qPCR Units can be positioned therein

3. Microcentrifuge capable of 12,000×g and Rotors 24×1.5/2.0 mL tubes. This configuration allows for 48 tubes to be processed at once

4. RNase-Free pipet tips with aerosol barrier for beta mercaptoethanol (beta-ME)

a. Filtered Pipette Tips, Sterile, 0.1-10 μl, 31 mm (Isolations)

b. Non-Filtered Pipette Tips, Sterile, 300 μl (Isolations)

c. Filtered Tips, Sterile, 1,000 μl Low Retention (Isolations)

d. 1-10 μL Microtips, racked (Isolations)

5. Pipettes are be packaged to accommodate isolations

6. Vortexers—Mixers with heads

7. Heating block, digital dry bath

8. Thermometer—Non-Mercury

9. Thermomixer—Multi-function Smart Mixer, mixes, shakes & vortex's per machine 2 ea

10. Ice Bucket—Open platform ice-free benchtop cooler/cool Rack PCR module

11. Disposable gloves—Powder-free M, L, XL

12. Biohazard waste bin w/biohazard bag with 7-10 gallon bags, 200 bags per carton

13. Bench coat paper

14. PCR Tubes

a. 0.2 mL-0.5 mL RNase, DNase free, sterile

b. Tube strips

15. Holders—Tubes—Microcentrifuge tube rack(s), reversible

16. PPE—As Needed

The mobile testing lab can be designed to be attachable for transfer to a powered vehicle such as a truck or other tall vehicle. The apparatus includes a trailer chassis assembly including a trailer frame and a trailer hitch assembly mounted to the trailer frame and adapted to be attached to a powered vehicle such as a truck or the like for facilitating transport thereof. A plurality of wheels are movably mounted to the trailer frame and extends therebelow to facilitate this transport movement.

The apparatus further includes a trailer housing including a trailer floor mounted on the trailer frame and a plurality of trailer walls, preferably a front wall, left wall, right wall and rear wall which extend generally upwardly from the trailer floor. A trailer door opening is defined in the trailer walls and especially defined in the rear wall and includes doors movable extending thereover preferably. A trailer roof is included extending between the individual trailer walls at a position above the trailer floor to define therebetween an interior chamber. Also an uppermost chamber area is defined in the uppermost area of the interior chamber for the purpose of facilitating the gathering of hydrogen gases therein which are lighter than air.

A door assembly is preferably attached to the trailer walls and is selectively positionable between a closed position and an opened position to facilitate access to the interior chamber through the trailer door opening. A plurality of electrical power supplies are positioned within the interior chamber for the purpose of supplying electrical power at remote locations when the trailer is moved to such locations.

One embodiment uses hydrogen fuel to generate electrical power. These electrical power supplies can give off hydrogen gas which the construction of the present invention is designed specifically to accumulate and expel from the interior chamber of the housing. A hydrogen gas ventilation assembly is included for selectively exhausting of hydrogen gases from the interior chamber into the external ambient environment. This hydrogen gas ventilation assembly is responsive to receiving an emergency exhausting signal from the hydrogen gas sensing device to activate and exhaust hydrogen gases from the interior chamber into the exterior ambient environment. At least one charging status display and control panel is electrically connected to the electrical power supply for monitoring the status and for controlling operations thereof. The charging status display panels are mounted in the trailer walls and are positioned such as to be viewable as well as controllable by a user positioned outside of the trailer wall.

Advantages of the mobile testing labs may include one or more of the following. The pandemic has seen system enables many drive-through-testing sites so that testing is available locally. These mobile labs can be scaled up in production to provide sufficient testing sites. The mobile labs can be placed in underserved areas and go out to high-risk communities like nursing-home residents and homeless people who need testing. Other advantages may include:
Rapid Deployment
High Throughput
Accurate Results
Low to Moderate Cost
Turn-key Simple Solution
Scalable with post-COVID or communicable disease infrastructure value
Consistent and Reliable Execution
Logistics Support Nationally & Internationally
ACT, transparent accreditation of test kits for full life cycle w/results data
Integration with AIM and ACT, both gateways for future network
solutions that help with COVID or communicable disease right now (NFP initial deployment)
Community dynamic that allows for a more engaged population
Accurate Results with new premier industry reporting methods
Low to Moderate initial cost and a continuous cost reduction as scaled
Upgrades are simple as new tech and testing methods get released into the market

These and other features and advantages will become apparent from the following detailed description of illustrative embodiments thereof, which is to be read in connection with the accompanying drawings.

### BRIEF DESCRIPTION OF THE DRAWINGS

While the invention is particularly pointed out and distinctly described herein, a preferred embodiment is set forth in the following detailed description which may be best understood when read in connection with the accompanying drawings, in which:

FIGS. 1A-1C show exemplary systems to provide pandemic testing and certification for a large population.

FIGS. 2A-2H show exemplary processes to provide a mobile paper or digital certificate/passport for a person being tested for a pathogen.

FIG. 3A shows an exemplary process for testing a person for COVID and reporting the results in a QR code.

FIG. 3B shows in more detail step 1 of FIG. 3A.

FIG. 3C shows in more detail step 2 or FIG. 3A.

FIG. 3D illustrates step 4 of FIG. 3A.

FIG. 3E show in more detail step 7 of FIG. 3A.

FIG. 3F shows an exemplary test history for an employee.

FIG. 3G shows an exemplary mobile app with a 2D bar code that can be scanned to indicate whether a person is healthy or not.

FIGS. 4A-4D show exemplary details of a mobile testing lab.

### DETAILED DESCRIPTION OF THE PREFERRED EMBODIMENT

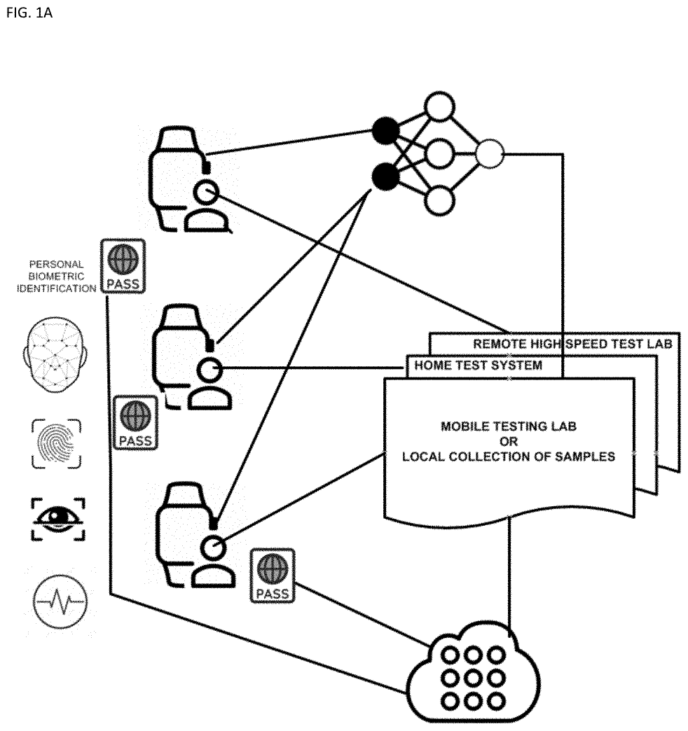

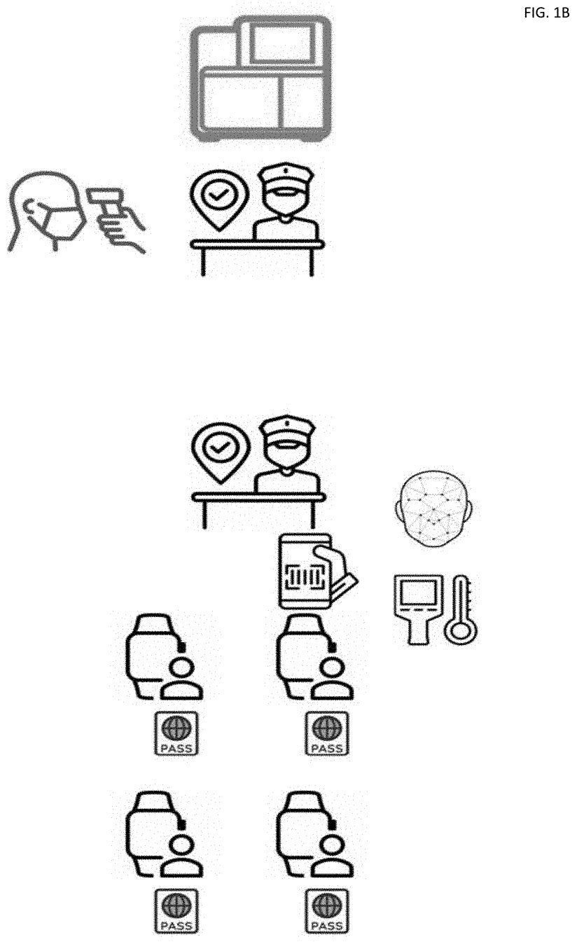

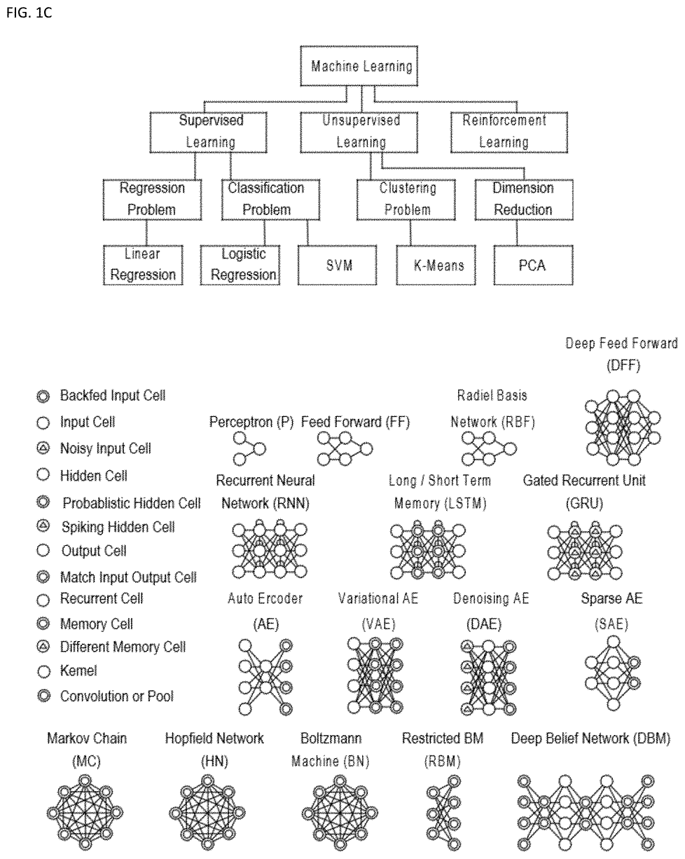

FIG. 1A shows an exemplary pathogen test system for individuals in a population that stamps a test result into a travel passport, while FIG. 1B shows an exemplary travel or work arrangement using the passport of FIG. 1A.

Turning now to FIG. 1A, one embodiment tests for COVID viruses. The user can be tested at home using a home kit. To reduce fraudulent testing, a face recognition system can be used for home testing where the camera in the face recognition system confirms continuity from the time the user inserts the swabs in the nose/mouth to the insertion of such test materials into the test machine to avoid fraudulent substitution of test materials into the test equipment. The user can also be tested at a mobile testing lab next to an office for volume testing. For population level testing, the user samples can be mailed to a remote high-speed testing lab.

User habits, temperature, heart rate and proximity to other users can be monitored by a wearable device such as a watch. In one embodiment, based on the personal data, a neural network or learning machine can predict the likelihood of COVID exposure in advance of running the lab tests. The learning system can direct the samples to be pooled for high speed testing, or can direct the samples for individual confirmation if the system expects the user to be carrying COVID virus. In this manner, testing efficiency is increased yet the cost is cheap. The result is provided to a web application server, which in turn securely stores the test results and updates the user's mobile passport which can be presented during travel or entrance to a workspace.

The user mobile device can be a phone, and a wearable device can be used to monitor vital signs. For example, wearable devices can spot COVID-19, the flu and other illnesses—even seeing if they can function as a personal early-detection system to contain the virus. They take wearable sensor data from both healthy people and those afflicted by COVID, compare and look for patterns in the data, then create artificial intelligence that could alert others whose own data patterns point to trouble. The system monitors fluctuations in key metrics, such as heart rate and respiration, days before symptoms. Flagged users could be instructed to quarantine and then, if symptoms appear, confirm with a test. One home test embodiment looks for a genetic signature in a saliva sample. The sample is put into a container with enzymes that, when heated, turn the liquid red to indicate no virus present or yellow to show an infection. In another embodiment, the user swabs her nose/throat and inserts the swabs into a home PCR tester. The results can be captured by mobile device and upload to the cloud server to generate the passport.

For home testing, the kit provides an answer in a short time such as in 15 minutes, and the test kits will be used when the wearable sensors detect abnormal patterns indicative of COVID. The wearable can detect temperature. The problem with temperature is more than half of people who have COVID-19, people with a bona fide infection—they never mount a fever response. Tracking fevers in aggregate, however, can help. Roughly a third of COVID or communicable disease-19 infections are asymptomatic, but a change in heart rate might still indicate one. If the resting heart rate has never gone up indicates that there's nothing going on, and that those who have COVID or communicable disease-19 can have an elevated resting heart rate about three days before symptoms. Others have found that resting heart-rate elevation, decreased physical activity and increased sleep were, in combination, a good signal for detecting the flu. Since an elevated heart rate or a drop in activity can be caused by lots of things, the system focuses on specific COVID or communicable disease-19 symptoms. A low blood-oxygen level, something in the low 90% range, or even dipping into the 80s, can be a signal for the severity of COVID or communicable disease-19. One embodiment of the wearable device measures temperature, heart rate, body motion and various other things, including chest wall movements and respiratory sounds for coughs. For some COVID or communicable disease-19 patients admitted in hospitals, coughing rates can reach an average of 100 per hour. The watch or phone can monitor recordings of COVID or communicable disease-19 coughs to see if it is possible for a smartphone to identify a unique signature. Routine screening of people who don't know they have COVID-19 could transform the fight against the disease. With COVID-19, people are most contagious in the few days before they develop symptoms and as symptoms first develop. Screening at home, maybe once or twice a week, would allow people to test themselves before going to work or school, getting on an airplane, attending an event, or visiting an elderly relative. Letting people know they are infectious in real-time would enable them to self-quarantine, and it would allow others to go about day-to-day life without risk of infecting others. The system conforms to the FDA template that spells out how a sample is to be collected and analyzed and how results are to be shown to a user without the need to send a sample to a lab for analysis. The FDA template also outlines how accurate the tests must be, with slightly lower standards than lab-based tests. People are contagious only when there's an extremely high virus level in their body, which can be detected by a less sensitive home test. An infection that goes undetected by a less-sensitive test would be caught a few days later when the person is tested again. Or the person would already be on the way to recovery and probably wouldn't be contagious.

To address communicable disease such as COVID risk prediction with person-to-person spread of risk factors, the embodiments described herein can employ metric learning corresponding to at least one “infectious” risk factor associated with contact tracing to generate an adjacency matrix for the at least one “infectious” risk factor. Then, a machine learning technique can monitor spread of COVID or communicable disease and contact tracing and vital signs from the person to predict risk of COVID exposure for the user. In one embodiment, a graph-based supervised learning, can be used, where labels can be smoothed over a graph by using graph-based regularization. Weighting factors can be calculated according to the number of the adjacency matrix.

Contact tracing can be done using the wearable or mobile devices. For example, Google and Apple has specified an Exposure Notification system in service of privacy-preserving contact tracing using Bluetooth signals and encryption in the notification. The Exposure Notification makes it possible to combat the spread of the coronavirus—the pathogen that causes COVID-19—by alerting participants about possible exposure to someone they have recently been in contact with, who has subsequently been positively diagnosed as having the virus. The Exposure Notification Service is the vehicle for implementing exposure notification and uses the Bluetooth Low Energy wireless technology for proximity detection of nearby smartphones, and for the data exchange mechanism. Exposure Notification Service uses the Bluetooth Low Energy service for detecting device proximity. It uses a Temporary Exposure Key—A key that's generated every 24 hours for privacy consideration. The result is a Diagnosis Key—The subset of Temporary Exposure Keys uploaded when the device owner is diagnosed as positive for the coronavirus. A Rolling Proximity Identifier which is a privacy preserving identifier derived from the Temporary Exposure Key can be sent in the broadcast of the Bluetooth payload. The identifier changes about every 15 minutes to prevent wireless tracking of the device. An Associated Encrypted Metadata (AEM) is a privacy preserving encrypted metadata that shall be used to carry protocol versioning and transmit (Tx) power for better distance approximation. The Associated Encrypted Metadata changes about every 15 minutes, at the same cadence as the Rolling Proximity Identifier, to prevent wireless tracking of the device.

As illustrated in FIG. 1B, upon traveling or going to work, the user can encounter two lines: a slow line for people without the proper credentials in a passport, or a fast lane for those who have pre-tested and the results recorded in their passports. The passports can be paper or electronic based. For those who travel untested, they go into a slow line where they are tested for temperature and swabs can be taken and then tested by a genetic sequencer. This process may require 1-4 hour delay for the user. Alternatively, if the user has been pretested and can show the testing in a passport, the user can be scanned, authenticated by facial camera and their personal mobile device, and be cleared to continue in a matter of minutes. Minimal additional testing can be done on the pretested, such as a thermal temperature check and facial check to see if they are in any contact tracing database that requires further check. In this manner, the system can be fast, low cost, and accurate enough for population level control of pandemics.

One embodiment provides a tunnel that sprays any sanitizing substance in a concentrated or in a diluted formulation. The spray can be performed over people in a tunnel or travel path through a series of showerheads to provide fine mists over travelers in the tunnel or pathway. One embodiment sprays a disinfectant using an electrostatic sprayer. The sprayer provides an electrical charge to solutions, allowing them to wrap conductive surfaces and even coverage. Another embodiment can use a silicone quaternary solution as a disinfectant/sanitizer such as Zetrisil, among others. The solution kills 99.99% of bacteria, fungus and viruses which are negatively charged nano particles. The positively charged disinfectant and/or sanitizer is attracted to the negatively charged bacteria/viruses. The agglomeration of the bacteria or virus particles and the disinfectant or sanitizer solution improves the efficacy of the disinfectant or sanitizer. Alternatively, by changing the magnetic charge around the bacteria or virus' cell structure, the sanitizer can damage the cell wall membrane causing osmosis imbalance to the bacteria or virus to lyse or rupture the cell wall.

FIG. 1C shows various learning machines or artificial neural networks that can predict COVID. The system has great predictive capabilities even when working with noisy, complex, and incomplete datasets, which may also be useful for modeling other viruses with complex epidemiology, such as the Zika virus. Inputs to the learning machine can include socioeconomic, behavioral, environmental, topographic, and demographic factors, together with mortality rates of preexisting conditions. For example, demographic (i.e., % male above 65), socioeconomic (i.e., median household income), and environmental factors (i.e., maximum terrain slope and precipitation) are influential in predicting COVID-19 incidence, indicating that the disease is not merely affected or driven by physiological conditions. The deep neural networks can capture potential non-linearity in the relationship between dependent and independent variables by stacking two or more hidden layers. With the obtained accuracy (age-adjusted), mortality rates of ischemic heart disease, pancreatic cancer, leukemia, Hodgkin's disease, mesothelioma, and cardiovascular disease, together with two socioeconomic and environmental factors (median household income and total precipitation), could contribute with the disease incidence.

In another embodiment, the learning machine checks for patterns in COVID or communicable disease's eruptions due to a lack of social distancing and can notify users to avoid risks from COVID. For example, the one or more entities can include one or more electronic devices associated with the person (e.g., personal computer, tablet, smartphone, Internet of Things (IoT) device). This can allow the person to be actively informed of the COVID or communicable disease at risk of being developed based on the risk factors. Such knowledge can motivate the person to make positive changes to mitigate the effects of the risk factors and the development of the COVID or communicable disease. Data related to the alert or message can be used to create and/or update one or more databases associated with the person.

As another example, the one or more entities can include one or more medical providers (e.g., doctors) associated with the person. For instance, an alert or message regarding the risk of developing COVID or communicable disease can be linked to the person's medical records. Such an alert can notify the medical provider(s) to monitor the person for the development of the COVID or communicable disease. In addition, such an alert can also be used by the medical provider(s) to perform testing on the person to identify any possible symptoms of the COVID or communicable disease, and to initiate a course of treatment in response to identifying any symptoms of the COVID or communicable disease. Data related to the alert or message can be used to create and/or update one or more databases associated with the one or more medical providers associated with the person, thereby allowing the one or more medical providers to maintain real-time updates of the person's medical history.

As yet another example, if the person is determined to be at risk of developing an COVID or communicable disease, the one or more medical insurances provider can use this information in the calculation of a premium for the person based on a likelihood of developing the COVID or communicable disease in the future. Data related to the alert or message can be used to create and/or update one or more databases associated with the one or more medical insurance providers associated with the person, thereby allowing the one or more medical insurance providers to maintain real-time updates of the person's medical history.

In one implementation, a graph associated with person-to-person links is received. In one embodiment, the graph includes a social network graph. The graph includes nodes and edges. For example, in one embodiment, each of the nodes can represent a person with a feature vector and COVID or communicable disease onset label, and each of the edges can represent a person-to-person link with a corresponding relationship vector. Next, one or more risk factors associated with the COVID or communicable disease are detected based on the graph. The one or more risk factors can be detected using a detector that can be implemented to search electronic documents (e.g., electronic versions of academic or medical papers) via one or more electronic databases or repositories, and identify the one or more risk factors from one or more electronic documents. One example of a website that can be used to detect one or more risk factors is PubMed, which is a free search engine maintained by the United States National Library of Medicine (NLM) at the National Institutes of Health (NIH) that can access one or more electronic databases (e.g., the Medical Literature Analysis and Retrieval System Online (MEDLINE) database). A relevant keyword search (e.g., a search for “factors spread in large social network”) can be performed to identify results for extraction using one or more data mining techniques. For example, if “COVID” and “loneliness” are included in the results of the keyword search, “COVID” and “loneliness” can be extracted from the results. Various data mining techniques can be used to mine data. Illustrative, queries can be passed to a website (e.g., PubMed) and the response can then be processed directly in a corresponding programing environment. The code can be automated to build systematic queries with different keywords. For each risk factor, a data structure for compactly representing the graph is generated to compute at least one person-to-person distance is generated. In one embodiment, the at least one person-to-person distance is computed based on features vectors of at least two persons and their corresponding relationship vector. The data structure can illustratively include an adjacency matrix or a suitable data structure for representing the graph for use by a computer to perform machine learning and/or artificial intelligence tasks. Metric learning is performed to calculate at least one person-to-person distance based on the adjacency matrix and, and a machine learning technique is performed on the graph with regularization of the at least one person-to-person distance. The machine learning technique can be performed for the nodes of the graph with risk factors and COVID or communicable disease onset labels. In one embodiment, the machine learning technique includes a supervised learning technique. One or more actions can be triggered in response to determining that a person is at risk of developing a COVID or communicable disease. In one embodiment, the system can send one or more alerts or messages that the person is at risk of developing a COVID or communicable disease, for example.

Pooled testing, guided by machine-learning algorithms, speed up testing. In pooled testing, many people's samples are combined into one. If no virus is detected in the combined sample, that means no one in the pool is infected. The entire pool can be cleared with just one test. However, if anyone in the pool is infected, the test will be positive and more testing will be required to figure out who has the virus. In one embodiment, using passport data, wearable device vital sign data, publicly available data from employers and schools, epidemiological data on local infection and testing rates, and data on travel patterns, social contacts, or sewage data, modelers can predict anyone's risk of having COVID-19 on a day-by-day basis. Accounting for the virus's spread between people and, therefore, for risk that is correlated. Using machine learning to model social networks has been a growing focus for researchers in computer science, economics, and other fields. Such algorithms, combined with data on jobs, classrooms, university dorms, and many other settings, allow machine-learning tools to estimate the potential that different people will interact. Knowing this likelihood can make group testing even more powerful. Machine learning provides precise individual-level estimates to make pooling work even at high prevalences, by identifying those likely to test positive and keeping them out of large pools. This allows highly flexible approaches to pooling that drive huge efficiency gains.

The learning machine can also predict risks for each person. For example, age is an indicator of whether someone is likely to die from the coronavirus, and the risk increases sharply among those over 80. Men were more likely to die than women of the same age: they accounted for 60% of all deaths. The learning system can identify groups with underlying medical conditions like obesity, diabetes, severe asthma, and cardiovascular disease were at higher risk, as were people with lower incomes. Black and South Asian people, as well as those from other ethnic minority groups, were more likely to die than white patients.

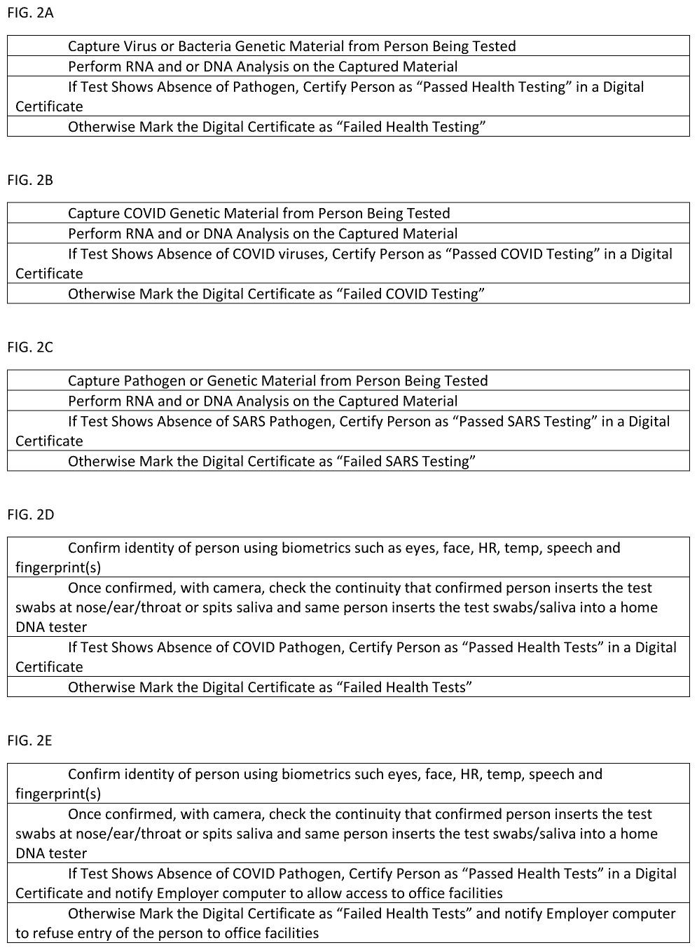

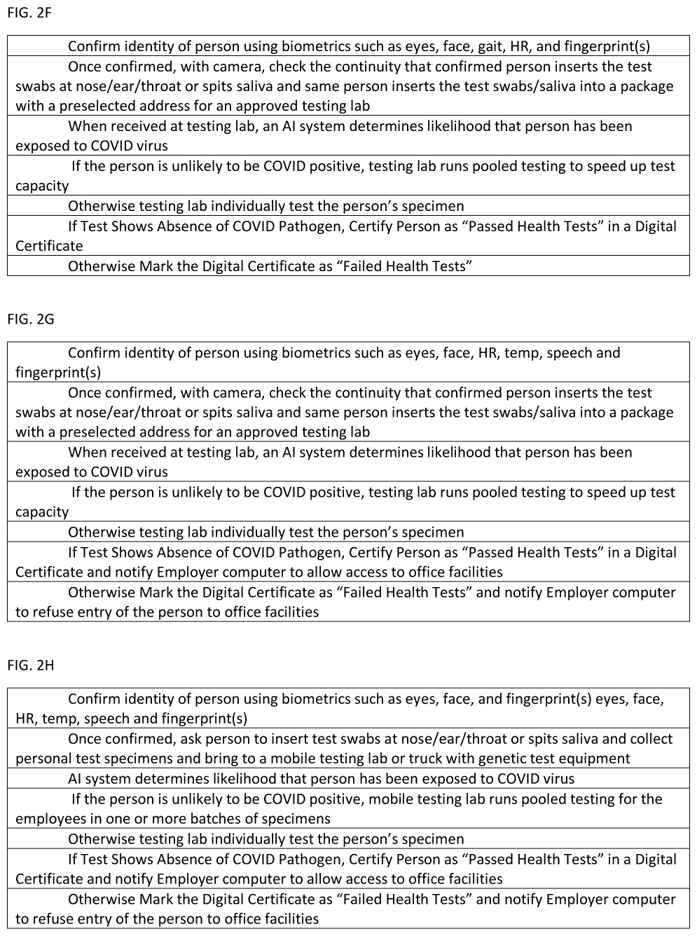

FIG. 2A shows an exemplary process to provide a mobile paper or digital certificate/passport for a person. The process includes:
Capture Genetic Material from Person Being Tested
Perform RNA and or DNA Analysis on the Captured Material
If Test Shows Absence of Pathogen, Certify Person as “Passed Health Testing” in a Digital Certificate
Otherwise Mark the Digital Certificate as “Failed Health Testing”

FIG. 2B shows an exemplary process to provide a mobile paper or digital certificate/passport for a person. The process includes:
Capture COVID Genetic Material from Person Being Tested
Perform RNA and or DNA Analysis on the Captured Material
If Test Shows Absence of COVID viruses, Certify Person as “Passed COVID Testing” in a Digital Certificate
Otherwise Mark the Digital Certificate as “Failed COVID Testing”

FIG. 2C shows an exemplary process to provide a mobile paper or digital certificate/passport for a person. The process includes:
Capture Genetic Material from Person Being Tested
Perform RNA and or DNA Analysis on the Captured Material
If Test Shows Absence of SARS Pathogen, Certify Person as “Passed SARS Testing” in a Digital Certificate
Otherwise Mark the Digital Certificate as “Failed SARS Testing”

FIG. 2D shows an exemplary process to provide a mobile paper or digital certificate/passport for a person. The process includes:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a home DNA tester
If Test Shows Absence of COVID Pathogen, Certify Person as “Passed Health Tests” in a Digital Certificate
Otherwise Mark the Digital Certificate as “Failed Health Tests”

FIG. 2E shows an exemplary process to provide a mobile paper or digital certificate/passport for a person. The process includes:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a home RNA and or DNA tester
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities

FIG. 2F shows an exemplary process to provide a mobile paper or digital certificate/passport for a person. The process includes:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a package with a preselected address for an approved testing lab
When received at testing lab, an AI system determines likelihood that person has been exposed to COVID virus
If the person is unlikely to be COVID positive, testing lab runs pooled testing to speed up test capacity
Otherwise testing lab individually test the person's specimen
If Test Shows Absence of COVID Pathogen, Certify Person as “Passed Health Tests” in a Digital Certificate
Otherwise Mark the Digital Certificate as “Failed Health Tests”

FIG. 2G shows an exemplary process to provide a mobile paper or digital certificate/passport for a person. The process includes:
Confirm identity of person using biometrics such as eyes, face, and fingerprint(s)
Once confirmed, with camera, check the continuity that confirmed person inserts the test swabs at nose/ear/throat or spits saliva and same person inserts the test swabs/saliva into a package with a preselected address for an approved testing lab
When received at testing lab, an AI system determines likelihood that person has been exposed to COVID virus
If the person is unlikely to be COVID positive, testing lab runs pooled testing to speed up test capacity
Otherwise testing lab individually test the person's specimen
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities

FIG. 2H shows an exemplary process to provide a mobile paper or digital certificate/passport for a person. The process includes:
Confirm identity of person using biometrics such as eyes, face, and fingerprint(s)
Once confirmed, ask person to insert test swabs at nose/ear/throat or spits saliva and collect personal test specimens and bring to a mobile testing lab or truck with genetic test equipment
AI system determines likelihood that person has been exposed to COVID virus
If the person is unlikely to be COVID positive, mobile testing lab runs pooled testing for the employees in one or more batches of specimens
Otherwise testing lab individually test the person's specimen
If Test Shows Absence of COVID Pathogen, Certify Person as “Passed Health Tests” in a Digital Certificate and notify Employer computer to allow access to office facilities
Otherwise Mark the Digital Certificate as “Failed Health Tests” and notify Employer computer to refuse entry of the person to office facilities.

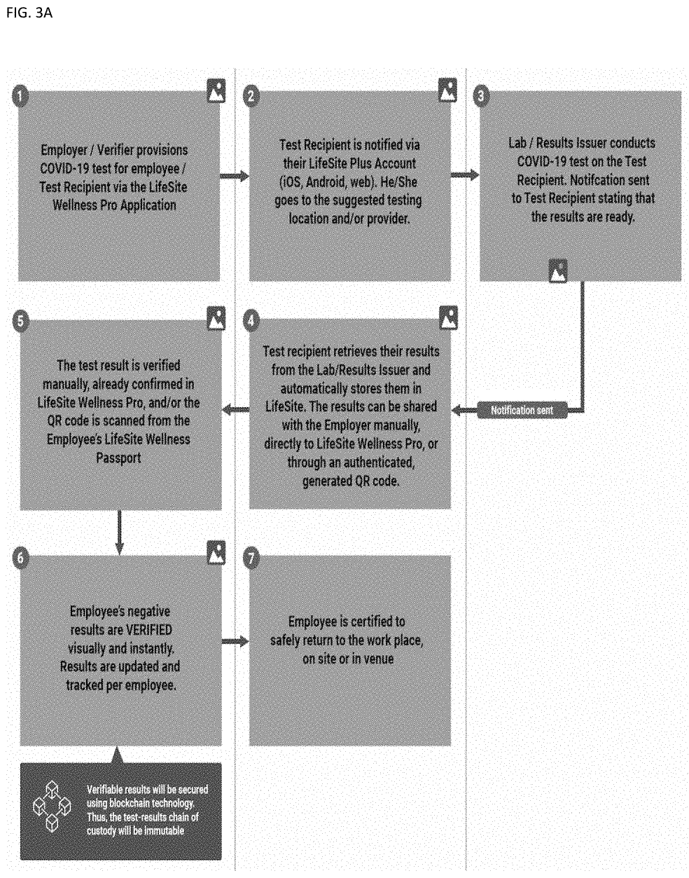

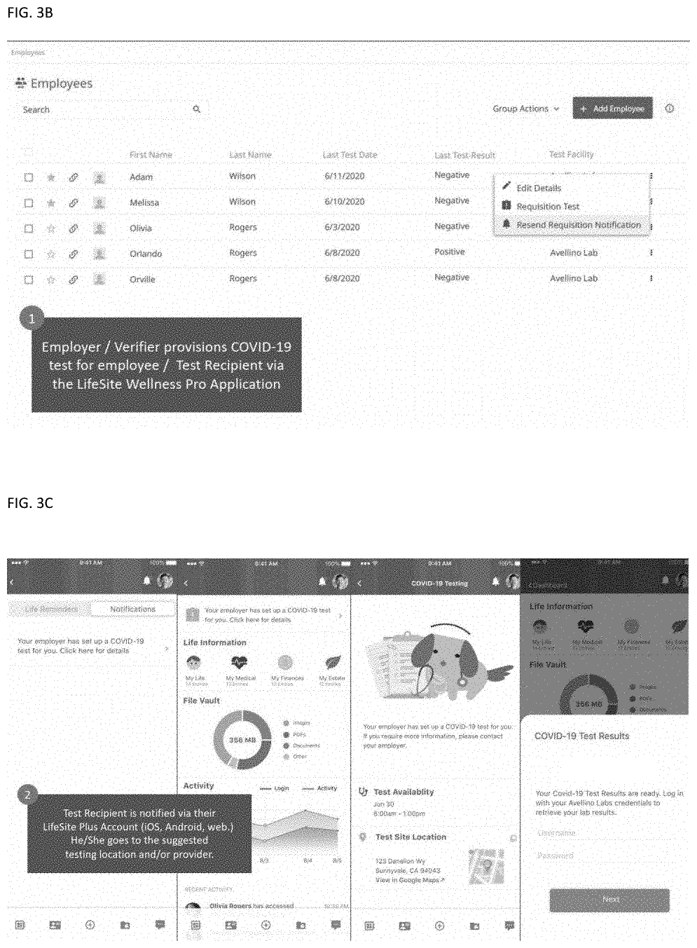

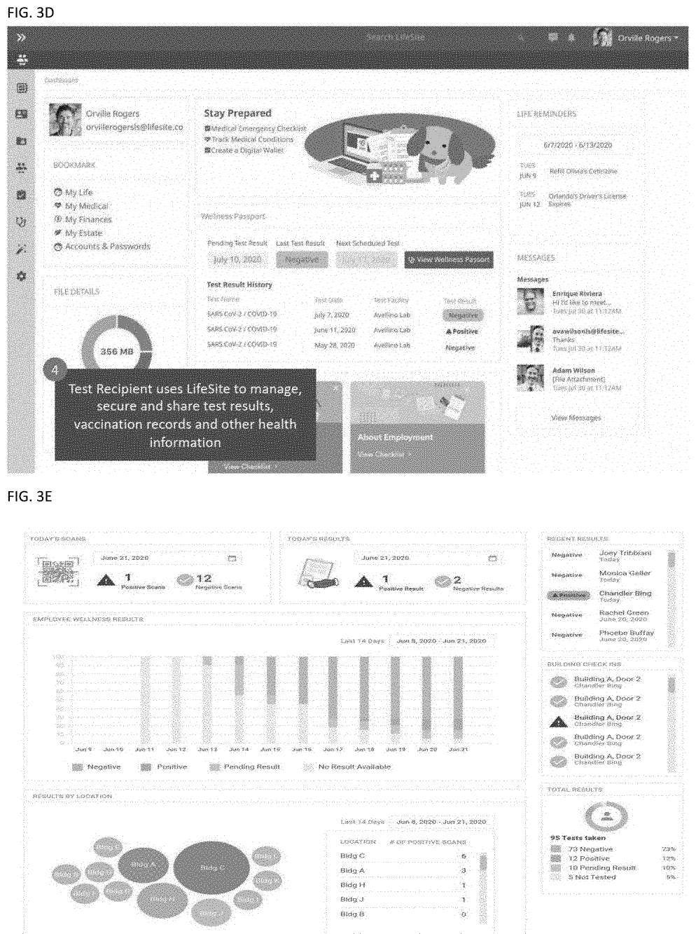

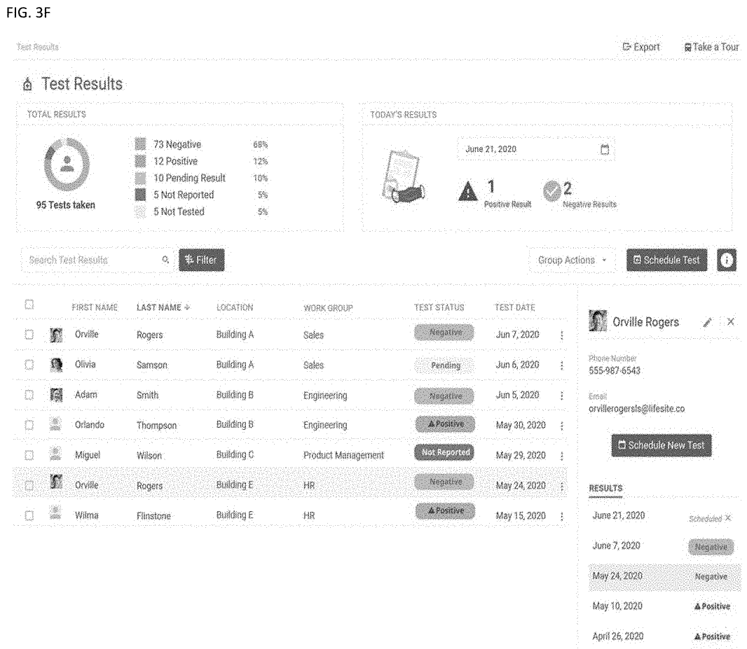

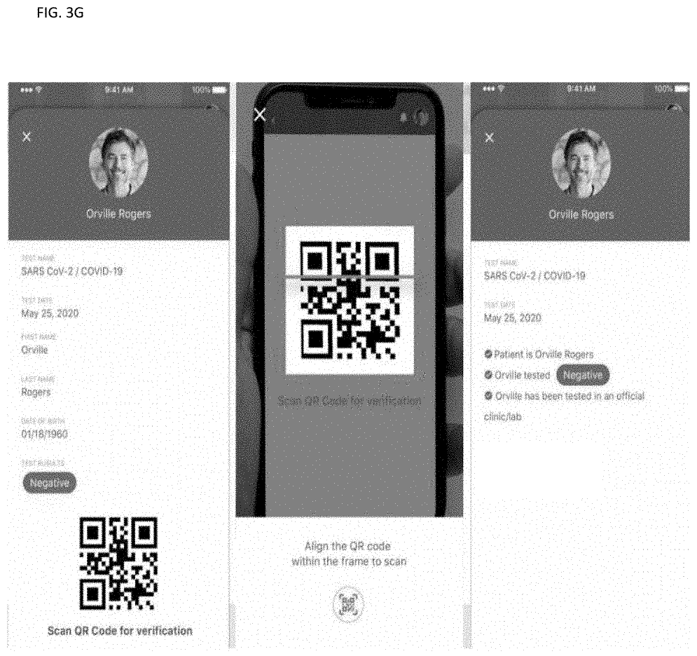

FIG. 3A shows an exemplary process for testing a person for COVID and reporting the results in a QR code. FIG. 3B shows in more detail step 1 of FIG. 3A; FIG. 3C shows in more detail step 2 or FIG. 3A; FIG. 3D illustrates step 4 of FIG. 3A; FIGS. 3E-3F show in more detail step 7 of FIG. 3A; FIG. 3G shows an exemplary mobile app with a 2D bar code that can be scanned to indicate whether a person is healthy or not; and FIG. 3H shows an exemplary test history for an employee. Referring to FIGS. 3A-3H collectively, in step 1 an employer or verifier provisions a COVID-19 test for and employee or a test recipient using a web-based application or mobile app. In step 2 the test recipient is notified in his/her account from an application such as a mobile app. The recipient travels to the suggested testing location. In Step 3, the lab or the result issuer conducts the COVID-19 tests on the test recipient. When done, a notification is sent to the test recipient stating that the results are ready. In step 4 the test recipient retrieves the result from the lab results issuer and automatically stores the result in a secure medical history database. The results can be shared with the employer manually, directly to a server app, or through an authenticated QR code that is generated, all steps are secured and encrypted. In step 5, the test result is verified manually and confirmed in the mobile app. Alternatively, the QR code can be scanned from the employers' wellness passport information. In step 6, negative COVID results can be verified visually and instantly and the results are tracked and updated per employee. In step 7, the employee is certified to safely return to the workplace on site or at a selected venue. In one embodiment verifiable results can be secured using blockchain and the test result chain of custody will be immutable.

The system provides customizable permissions that allow specific information to be securely shared with trusted sources. Multiple layers of privacy and security protocols constantly keep information encrypted and accessible. A central, organized vault is provided for test results and vaccination records. The user has mobile access with secure, convenient access to test results. The system can quickly add new results with an encrypted scanner.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models. Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

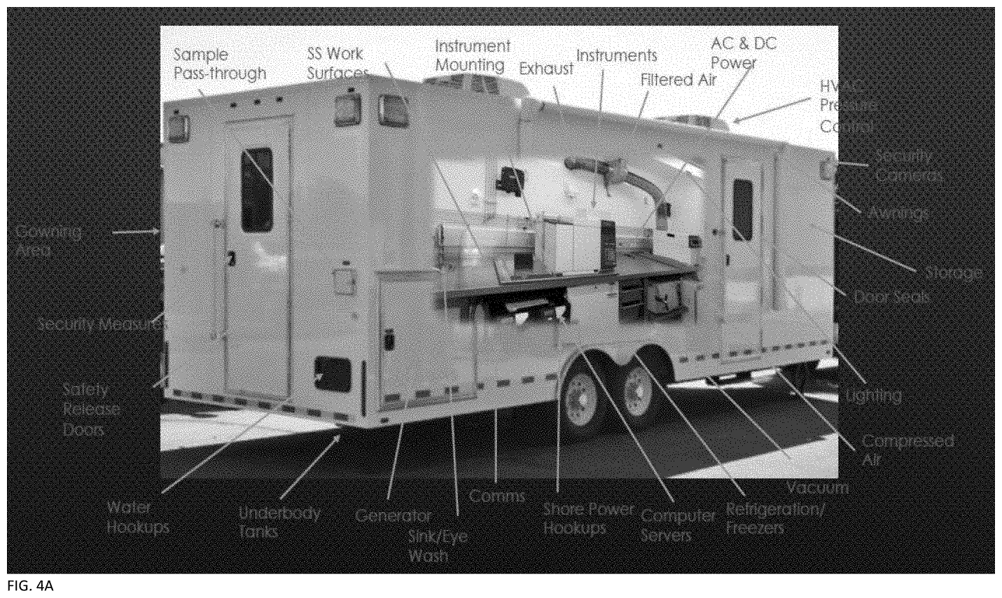

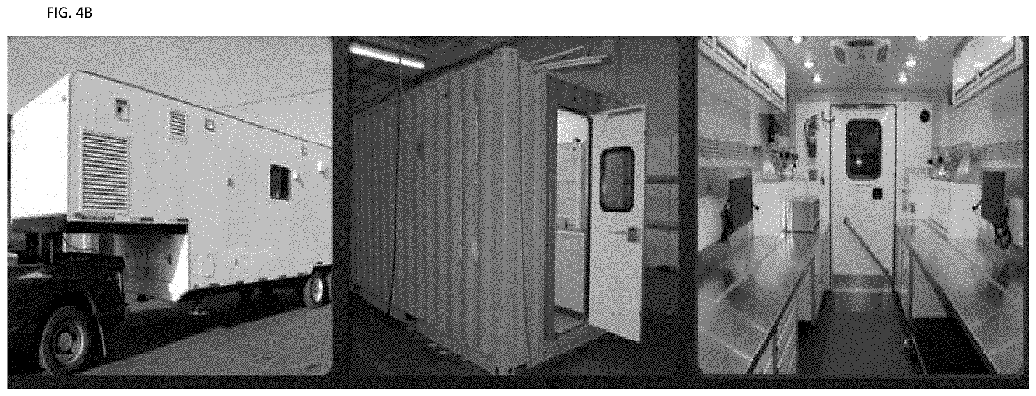

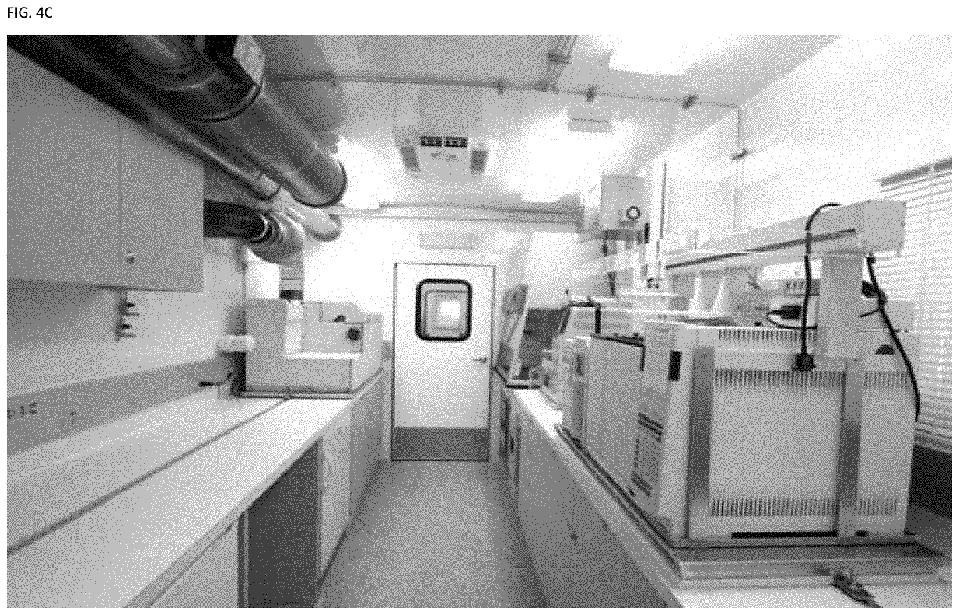

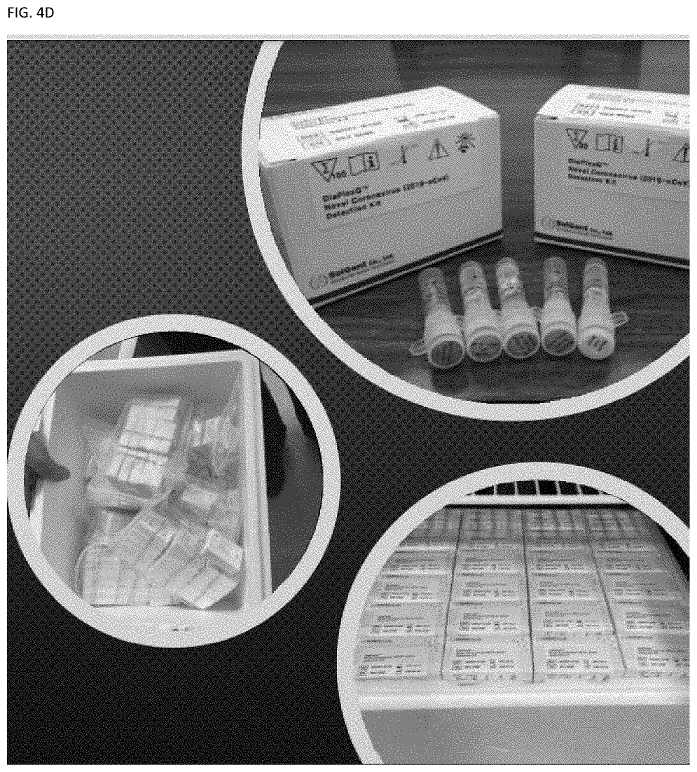

FIG. 4A shows an exemplary mobile testing laboratory (lab). The mobile testing lab can be positioned as a closed, authenticated end-to-end solution near a building to support return to work for companies, schools, churches, amusement parks, etc. Other use cases may include:
Factories, work sites, processing plants, etc.
Use for sports leagues, staff and families to venues
Use for events, activities, stadiums, etc.
Support for highly sensitive environments ie. nursing homes and family/visitors
Cruise ships, camps, etc.

One embodiment includes two (2) workstations, each trailer will be equipped with −20C.° and 4C.° storage. In general, one person isolates, and one person performs PCR so contamination does not occur. Each of these individuals should be a scientist or trained technician.

One key to eventually scaling back self-isolation and quarantining and social distancing will be massively expanding the number of people who can get tested for the coronavirus up to the point where ideally you can test millions of people per day. The system can use test results from PCR, polymerase chain reaction, to amplify DNA. One embodiment performs massive testing by using the genome sequencing machines that are normally used to sequence human genomes. In this embodiment, thousands, tens of thousands, even millions of samples, could be pooled together and then read out on speedy sequencing machines. The mobile testing labs can collect the swabs into one place and send them to a sequencing center. The results are electronically communicated to people being tested to minimize the delay. In another embodiment, a home test is provided where everybody could use from home before he/she goes to work. Once a week, twice a week, you could test yourself, and have an idea whether you were infected and if you were, then you could, you would know what to do. You′d have to stay home, get in touch with public health authorities, call your contacts and let them know that you have this virus. So there's different ways in theory to do this at home testing. Another embodiment uses antigen testing. An antigen test is when you have an antibody that sticks to the virus. The antibody detects spike protein on the surface of the virus. A test strip with antibodies and that that is going to stick to that antigen if it is in your nasal swab. In another embodiment, a simplified genetic test that would combine the accuracy of PCR-based testing with the convenience of decentralized at-home testing. In another embodiment, after a person is tested, their status is anchored in a blockchain. Verified and certified test results are transmitted in the form of a QR code that can easily be presented and read via scan. The IT infrastructure, from the systems in the lab to the blockchain and the app, meets the highest privacy standards and significantly speeds up the delivery of test results.

Items Included in each Trailer for COVID or communicable disease testing:

1. Self-contained bio safety cabinet, no outside venting with HEPA filtration

2. qPCR Equipment—Minimum Throughput/trailer/hour 92 (greater throughput can be achieved with medium or hi-capacity units). A plurality of qPCR Units can be positioned therein

3. Microcentrifuge capable of 12,000×g and Rotors 24×1.5/2.0 mL tubes. This configuration allows for 48 tubes to be processed at once

4. RNase-Free pipet tips with aerosol barrier for beta mercaptoethanol (beta-ME)
a. Filtered Pipette Tips, Sterile, 0.1-10 μl, 31 mm (Isolations)
b. Non-Filtered Pipette Tips, Sterile, 300 μl (Isolations)
c. Filtered Tips, Sterile, 1,000 μl Low Retention (Isolations)
d. 1-10 μL Microtips, racked (Isolations)

5. Pipettes are be packaged to accommodate isolations

6. Vortexers—Mixers with heads

7. Heating block, digital dry bath

8. Thermometer—Non-Mercury

9. Thermomixer—Multi-function Smart Mixer, mixes, shakes & vortex's per machine 2 ea

10. Ice Bucket—Open platform ice-free benchtop cooler/cool Rack PCR module

11. Disposable gloves—Powder-free M, L, XL

12. Biohazard waste bin w/biohazard bag with 7-10 gallon bags, 200 bags per carton

13. Bench coat paper

14. PCR Tubes
a. 0.2 mL-0.5 mL RNase, DNase free, sterile
b. Tube strips

15. Holders—Tubes—Microcentrifuge tube rack(s), reversible

16. PPE—As Needed

One 20 ft unit runs continually all day, dependent on configuration of qPCR (2 small machines) can process 22,000 tests every 24 hours, with a 2 hour lead time. One 50 ft w/largest PCR machines available automation used, 97,000 test every 24 hrs. The mobile units will be stocked with masks, suits, sanitizer and other required safety equipment. The system can run a Nucleic Acid Based Molecular Diagnostic Test in one embodiment with the following steps:

1. Sample preparation

2. Nucleic acid extraction

3. Nucleic acid amplification (optional)

4. Detection

Many sample types are used for genetic analysis, such as blood, urine, sputum and tissue samples. The diagnostic test determines the type of sample required as not all samples are representative of the disease process. These samples have a variety of constituents, but usually only one of these is of interest. For example, in blood, high concentrations of erythrocytes can inhibit the detection of a pathogenic organism. Therefore a purification and/or concentration step at the beginning of the nucleic acid test is often required.

Blood is one of the more commonly sought sample types. It has three major constituents: leukocytes (white blood cells), erythrocytes (red blood cells) and thrombocytes (platelets). The thrombocytes facilitate clotting and remain active in vitro. To inhibit coagulation, the specimen is mixed with an agent such as ethylenediaminetetraacetic acid (EDTA) prior to purification and concentration. Erythrocytes are usually removed from the sample in order to concentrate the target cells. In humans, erythrocytes account for approximately 99% of the cellular material but do not carry DNA as they have no nucleus. Furthermore, erythrocytes contain components such as hemoglobin that can interfere with the downstream nucleic acid amplification process (described below). Removal of erythrocytes can be achieved by differentially lysing the erythrocytes in a lysis solution, leaving remaining cellular material intact which can then be separated from the sample using centrifugation. This provides a concentration of the target cells from which the nucleic acids are extracted.

The exact protocol used to extract nucleic acids depends on the sample and the diagnostic assay to be performed. For example, the protocol for extracting viral RNA will vary considerably from the protocol to extract genomic DNA. However, extracting nucleic acids from target cells usually involves a cell lysis step followed by nucleic acid purification. The cell lysis step disrupts the cell and nuclear membranes, releasing the genetic material. This is often accomplished using a lysis detergent, such as sodium dodecyl sulfate, which also denatures the large amount of proteins present in the cells.

The nucleic acids are then purified with an alcohol precipitation step, usually ice-cold ethanol or isopropanol, or via a solid phase purification step, typically on a silica matrix in a column, resin or on paramagnetic beads in the presence of high concentrations of a chaotropic salt, prior to washing and then elution in a low ionic strength buffer. An optional step prior to nucleic acid precipitation is the addition of a protease which digests the proteins in order to further purify the sample. Other lysis methods include mechanical lysis via ultrasonic vibration and thermal lysis where the sample is heated to 94° C. to disrupt cell membranes. The target DNA or RNA may be present in the extracted material in very small amounts, particularly if the target is of pathogenic origin. Nucleic acid amplification provides the ability to selectively amplify (that is, replicate) specific targets present in low concentrations to detectable levels.

The most commonly used nucleic acid amplification technique is the polymerase chain reaction (PCR). PCR is well known in this field and comprehensive description of this type of reaction is provided in E. van Pelt-Verkuil et al., Principles and Technical Aspects of PCR Amplification, Springer, 2008. PCR is a powerful technique that amplifies a target DNA sequence against a background of complex DNA. If RNA is to be amplified (by PCR), it must be first transcribed into cDNA (complementary DNA) using an enzyme called reverse transcriptase. Afterwards, the resulting cDNA is amplified by PCR. PCR is an exponential process that proceeds as long as the conditions for sustaining the reaction are acceptable. The components of the reaction are:

1. pair of primers—short single strands of DNA with around 10-50 nucleotides complementary to the regions flanking the target sequence

2. DNA polymerase—a thermostable enzyme that synthesizes DNA

3. deoxyribonucleoside triphosphates (dNTPs)—provide the nucleotides that are incorporated into the newly synthesized DNA strand

4. buffer—to provide the optimal chemical environment for DNA synthesis

PCR typically involves placing these reactants in a small tube (\tilde{ } 10-50 microlitres) containing the extracted nucleic acids. The tube is placed in a thermal cycler; an instrument that subjects the reaction to a series of different temperatures for varying amounts of time. The standard protocol for each thermal cycle involves a denaturation phase, an annealing phase, and an extension phase. The extension phase is sometimes referred to as the primer extension phase. In addition to such three-step protocols, two-step thermal protocols can be employed, in which the annealing and extension phases are combined. The denaturation phase typically involves raising the temperature of the reaction to 90-95° C. to denature the DNA strands; in the annealing phase, the temperature is lowered to \tilde{ } 50-60° C. for the primers to anneal; and then in the extension phase the temperature is raised to the optimal DNA polymerase activity temperature of 60-72° C. for primer extension. This process is repeated cyclically around 20-40 times, the end result being the creation of millions of copies of the target sequence between the primers.

There are a number of variants to the standard PCR protocol such as multiplex PCR, linker-primed PCR, direct PCR, tandem PCR, real-time PCR and reverse-transcriptase PCR, amongst others, which have been developed for molecular diagnostics.

Multiplex PCR uses multiple primer sets within a single PCR mixture to produce amplicons of varying sizes that are specific to different DNA sequences. By targeting multiple genes at once, additional information may be gained from a single test-run that otherwise would require several experiments. Optimization of multiplex PCR is more difficult though and requires selecting primers with similar annealing temperatures, and amplicons with similar lengths and base composition to ensure the amplification efficiency of each amplicon is equivalent.

Linker-primed PCR, also known as ligation adaptor PCR, is a method used to enable nucleic acid amplification of essentially all DNA sequences in a complex DNA mixture without the need for target-specific primers. The method firstly involves digesting the target DNA population with a suitable restriction endonuclease (enzyme). Double-stranded oligonucleotide linkers (also called adaptors) with a suitable overhanging end are then ligated to the ends of target DNA fragments using a ligase enzyme. Nucleic acid amplification is subsequently performed using oligonucleotide primers which are specific for the linker sequences. In this way, all fragments of the DNA source which are flanked by linker oligonucleotides can be amplified.

Direct PCR describes a system whereby PCR is performed directly on a sample without any, or with minimal, nucleic acid extraction. It has long been accepted that PCR reactions are inhibited by the presence of many components of unpurified biological samples, such as the haem component in blood. Traditionally, PCR has required extensive purification of the target nucleic acid prior to preparation of the reaction mixture. With appropriate changes to the chemistry and sample concentration, however, it is possible to perform PCR with minimal DNA purification, or direct PCR. Adjustments to the PCR chemistry for direct PCR include increased buffer strength, the use of polymerases which have high activity and processivity, and additives which chelate with potential polymerase inhibitors.

Tandem PCR utilizes two distinct rounds of nucleic acid amplification to increase the probability that the correct amplicon is amplified. One form of tandem PCR is nested PCR in which two pairs of PCR primers are used to amplify a single locus in separate rounds of nucleic acid amplification. The first pair of primers hybridize to the nucleic acid sequence at regions external to the target nucleic acid sequence. The second pair of primers (nested primers) used in the second round of amplification bind within the first PCR product and produce a second PCR product containing the target nucleic acid, that will be shorter than the first one. The logic behind this strategy is that if the wrong locus were amplified by mistake during the first round of nucleic acid amplification, the probability is very low that it would also be amplified a second time by a second pair of primers and thus ensures specificity.

Real-time PCR, or quantitative PCR, is used to measure the quantity of a PCR product in real time. By using a fluorophore-containing probe or fluorescent dyes along with a set of standards in the reaction, it is possible to quantitate the starting amount of nucleic acid in the sample. This is particularly useful in molecular diagnostics where treatment options may differ depending on the pathogen load in the sample.

Reverse-transcriptase PCR (RT-PCR) is used to amplify DNA from RNA. Reverse transcriptase is an enzyme that reverse transcribes RNA into complementary DNA (cDNA), which is then amplified by PCR. RT-PCR is widely used in expression profiling, to determine the expression of a gene or to identify the sequence of an RNA transcript, including transcription start and termination sites. It is also used to amplify RNA viruses such as human immunodeficiency virus or hepatitis C virus.

Isothermal amplification is another form of nucleic acid amplification which does not rely on the thermal denaturation of the target DNA during the amplification reaction and hence does not require sophisticated machinery. Isothermal nucleic acid amplification methods can therefore be carried out in primitive sites or operated easily outside of a laboratory environment. A number of isothermal nucleic acid amplification methods have been described, including Strand Displacement Amplification, Transcription Mediated Amplification, Nucleic Acid Sequence Based Amplification, Recombinase Polymerase Amplification, Rolling Circle Amplification, Ramification Amplification, Helicase-Dependent Isothermal DNA Amplification and Loop-Mediated Isothermal Amplification.

Isothermal nucleic acid amplification methods do not rely on the continuing heat denaturation of the template DNA to produce single stranded molecules to serve as templates for further amplification, but instead rely on alternative methods such as enzymatic nicking of DNA molecules by specific restriction endonucleases, or the use of an enzyme to separate the DNA strands, at a constant temperature.

Strand Displacement Amplification (SDA) relies on the ability of certain restriction enzymes to nick the unmodified strand of hemi-modified DNA and the ability of a 5′-3′ exonuclease-deficient polymerase to extend and displace the downstream strand. Exponential nucleic acid amplification is then achieved by coupling sense and antisense reactions in which strand displacement from the sense reaction serves as a template for the antisense reaction. The use of nickase enzymes which do not cut DNA in the traditional manner but produce a nick on one of the DNA strands, such as N. Alwl, N. BstNBI and Mlyl, are useful in this reaction. SDA has been improved by the use of a combination of a heat-stable restriction enzyme (Aval) and heat-stable Exo-polymerase (Bst polymerase). This combination has been shown to increase amplification efficiency of the reaction from 108 fold amplification to 1010 fold amplification so that it is possible using this technique to amplify unique single copy molecules.

Transcription Mediated Amplification (TMA) and Nucleic Acid Sequence Based Amplification (NASBA) use an RNA polymerase to copy RNA sequences but not corresponding genomic DNA. The technology uses two primers and two or three enzymes, RNA polymerase, reverse transcriptase and optionally RNase H (if the reverse transcriptase does not have RNase activity). One primer contains a promoter sequence for RNA polymerase. In the first step of nucleic acid amplification, this primer hybridizes to the target ribosomal RNA (rRNA) at a defined site. Reverse transcriptase creates a DNA copy of the target rRNA by extension from the 3′ end of the promoter primer. The RNA in the resulting RNA:DNA duplex is degraded by the RNase activity of the reverse transcriptase if present or the additional RNase H. Next, a second primer binds to the DNA copy. A new strand of DNA is synthesized from the end of this primer by reverse transcriptase, creating a double-stranded DNA molecule. RNA polymerase recognizes the promoter sequence in the DNA template and initiates transcription. Each of the newly synthesized RNA amplicons re-enters the process and serves as a template for a new round of replication.

In Recombinase Polymerase Amplification (RPA), the isothermal amplification of specific DNA fragments is achieved by the binding of opposing oligonucleotide primers to template DNA and their extension by a DNA polymerase. Heat is not required to denature the double-stranded DNA (dsDNA) template. Instead, RPA employs recombinase-primer complexes to scan dsDNA and facilitate strand exchange at cognate sites. The resulting structures are stabilised by single-stranded DNA binding proteins interacting with the displaced template strand, thus preventing the ejection of the primer by branch migration. Recombinase disassembly leaves the 3′ end of the oligonucleotide accessible to a strand displacing DNA polymerase, such as the large fragment of Bacillus subtilis Pol I (Bsu), and primer extension ensues. Exponential nucleic acid amplification is accomplished by the cyclic repetition of this process. Helicase-dependent amplification (HDA) mimics the in vivo system in that it uses a DNA helicase enzyme to generate single-stranded templates for primer hybridization and subsequent primer extension by a DNA polymerase. In the first step of the HDA reaction, the helicase enzyme traverses along the target DNA, disrupting the hydrogen bonds linking the two strands which are then bound by single-stranded binding proteins. Exposure of the single-stranded target region by the helicase allows primers to anneal. The DNA polymerase then extends the 3′ ends of each primer using free deoxyribonucleoside triphosphates (dNTPs) to produce two DNA replicates. The two replicated dsDNA strands independently enter the next cycle of HDA, resulting in exponential nucleic acid amplification of the target sequence.

Other DNA-based isothermal techniques include Rolling Circle Amplification (RCA) in which a DNA polymerase extends a primer continuously around a circular DNA template, generating a long DNA product that consists of many repeated copies of the circle. By the end of the reaction, the polymerase generates many thousands of copies of the circular template, with the chain of copies tethered to the original target DNA. This allows for spatial resolution of target and rapid nucleic acid amplification of the signal. Up to 1012 copies of template can be generated in 1 hour. Ramification amplification is a variation of RCA and utilizes a closed circular probe (C-probe) or padlock probe and a DNA polymerase with a high processivity to exponentially amplify the C-probe under isothermal conditions.

Loop-mediated isothermal amplification (LAMP), offers high selectivity and employs a DNA polymerase and a set of four specially designed primers that recognize a total of six distinct sequences on the target DNA. An inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. The cycling reaction continues with accumulation of 109 copies of target in less than an hour. The final products are stem-loop DNAs with several inverted repeats of the target and cauliflower-like structures with multiple loops formed by annealing between alternately inverted repeats of the target in the same strand.

After completion of the nucleic acid amplification, the amplified product must be analysed to determine whether the anticipated amplicon (the amplified quantity of target nucleic acids) was generated. The methods of analyzing the product range from simply determining the size of the amplicon through gel electrophoresis, to identifying the nucleotide composition of the amplicon using DNA hybridization.

Gel electrophoresis is one of the simplest ways to check whether the nucleic acid amplification process generated the anticipated amplicon. Gel electrophoresis uses an electric field applied to a gel matrix to separate DNA fragments. The negatively charged DNA fragments will move through the matrix at different rates, determined largely by their size. After the electrophoresis is complete, the fragments in the gel can be stained to make them visible. Ethidium bromide is a commonly used stain which fluoresces under UV light.

The size of the fragments is determined by comparison with a DNA size marker (a DNA ladder), which contains DNA fragments of known sizes, run on the gel alongside the amplicon. Because the oligonucleotide primers bind to specific sites flanking the target DNA, the size of the amplified product can be anticipated and detected as a band of known size on the gel. To be certain of the identity of the amplicon, or if several amplicons have been generated, DNA probe hybridization to the amplicon is commonly employed.

DNA hybridization refers to the formation of double-stranded DNA by complementary base pairing. DNA hybridization for positive identification of a specific amplification product requires the use of a DNA probe around 20 nucleotides in length. If the probe has a sequence that is complementary to the amplicon (target) DNA sequence, hybridization will occur under favorable conditions of temperature, pH and ionic concentration. If hybridization occurs, then the gene or DNA sequence of interest was present in the original sample.

Optical detection is the most common method to detect hybridization. Either the amplicons or the probes are labelled to emit light through fluorescence or electrochemiluminescence. These processes differ in the means of producing excited states of the light-producing moieties, but both enable covalent labelling of nucleotide strands. In electrochemiluminescence (ECL), light is produced by luminophore molecules or complexes upon stimulation with an electric current. In fluorescence, it is illumination with excitation light which leads to emission.

Fluorescence is detected using an illumination source which provides excitation light at a wavelength absorbed by the fluorescent molecule, and a detection unit. The detection unit comprises a photosensor (such as a photomultiplier tube or charge-coupled device (CCD) array) to detect the emitted signal, and a mechanism (such as a wavelength-selective filter) to prevent the excitation light from being included in the photosensor output. The fluorescent molecules emit Stokes-shifted light in response to the excitation light, and this emitted light is collected by the detection unit. Stokes shift is the frequency difference or wavelength difference between emitted light and absorbed excitation light.

ECL emission is detected using a photosensor which is sensitive to the emission wavelength of the ECL species being employed. For example, transition metal-ligand complexes emit light at visible wavelengths, so conventional photodiodes and CCDs are employed as photosensors. An advantage of ECL is that, if ambient light is excluded, the ECL emission can be the only light present in the detection system, which improves sensitivity.

Microarrays allow for hundreds of thousands of DNA hybridization experiments to be performed simultaneously. Microarrays are powerful tools for molecular diagnostics with the potential to screen for thousands of genetic diseases or detect the presence of numerous infectious pathogens in a single test. A microarray consists of many different DNA probes immobilized as spots on a substrate. The target DNA (amplicon) is first labelled with a fluorescent or luminescent molecule (either during or after nucleic acid amplification) and then applied to the array of probes. The microarray is incubated in a temperature controlled, humid environment for a number of hours or days while hybridization between the probe and amplicon takes place. Following incubation, the microarray must be washed in a series of buffers to remove unbound strands. Once washed, the microarray surface is dried using a stream of air (often nitrogen). The stringency of the hybridization and washes is critical. Insufficient stringency can result in a high degree of nonspecific binding. Excessive stringency can lead to a failure of appropriate binding, which results in diminished sensitivity. Hybridization is recognized by detecting light emission from the labelled amplicons which have formed a hybrid with complementary probes.

Fluorescence from microarrays is detected using a microarray scanner which is generally a computer controlled inverted scanning fluorescence confocal microscope which typically uses a laser for excitation of the fluorescent dye and a photosensor (such as a photomultiplier tube or CCD) to detect the emitted signal. The fluorescent molecules emit Stokes-shifted light (described above) which is collected by the detection unit. The emitted fluorescence must be collected, separated from the unabsorbed excitation wavelength, and transported to the detector. In microarray scanners, a confocal arrangement is commonly used to eliminate out-of-focus information by means of a confocal pinhole situated at an image plane. This allows only the in-focus portion of the light to be detected. Light from above and below the plane of focus of the object is prevented from entering the detector, thereby increasing the signal to noise ratio. The detected fluorescent photons are converted into electrical energy by the detector which is subsequently converted to a digital signal. This digital signal translates to a number representing the intensity of fluorescence from a given pixel. Each feature of the array is made up of one or more such pixels. The final result of a scan is an image of the array surface. The exact sequence and position of every probe on the microarray is known, and so the hybridized target sequences can be identified and analysed simultaneously.

For home testing kit, a lab-on-a-chip (LOC) device can perform genetic analysis of the COVID sample, the LOC device having an inlet for receiving the sample containing genetic material including DNA and RNA, a supporting substrate, a plurality of reagent reservoirs, a first nucleic acid amplification section for amplifying at least some of the genetic material, and, a second nucleic acid amplification section for amplifying at least some the genetic material in parallel with the first nucleic acid amplification section, wherein, the first nucleic acid amplification section and the second nucleic acid amplification section are both supported on the supporting substrate. More on this device is described in US Application 20110312730, the content of which is incorporated by reference.

The mobile testing lab is housed within a portable trailer to facilitate movement thereof to remote locations for providing power for various purposes including charging batteries at remote locations. One examplary apparatus includes a trailer housing with trailer wheels and a trailer hitch assembly to facilitate movement of the trailer by towing by a powered vehicle such as a truck. The construction of the trailer housing includes a front wall, along with a left side wall and a right side wall and a rear wall assembly defining a rear door opening therein with rear doors selectively extending thereover in a closed position and movable to an opened position to facilitate entry and exit by a user into the interior chamber. The left rear door is pivotally secured with respect to the left side wall 18, and the right rear door is pivotally secured with respect to the right-side wall to facilitate access to the interior chamber of the trailer housing 10 as needed. A trailer chassis includes a trailer frame which supports the trailer housing 10 and all portions thereof and defines a trailer floor on the upper surface thereof. The trailer roof extends across the upper portion of the trailer side walls and the front wall and the rear doors. One implementation has the following:
Enclosed tandem-axle bumper-pull ball hitch trailer
GVWR 14,000 lbs.
Electric trailer brakes
Stabilizing jacks on all four corners
One entry/exit door with window on the curb side and one at the rear
Steel frame with aluminum or fiberglass sides and roof
Wood floor covered in a non-porous, sealed material
Provision for fuel tank
Fresh and grey water tanks
Interior
Fiberglass/plastic or aluminum paneled interior walls
Foam insulated walls and ceiling
All seams sealed
Stainless steel benchtop surfaces
Stainless sink with eyewash
Painted metal or stainless storage cabinets and drawers under benchtop
Painted metal or stainless overhead metal storage cabinets and shelving
Overhead LED lighting (AC and DC)
Two workstations with leg-holes
Electrical
Shore power connectivity
Option for gas/diesel generator(s)
Breaker panel on interior
20 amp, 120V circuits—AC
12V DC lighting
Roof mounted air conditioner/heat: (2) at 15K BTU/5,600 W
Equipment (to be integrated/mounted)
Under the counter 4° C. refrigerator
Under the counter −20° C. freezer
Benchtop biosafety cabinet
Flammable safety cabinet

Battery banks are included within the interior chamber of the trailer housing. Such battery banks commonly generate gaseous hydrogen when providing electrical power and during charging thereof, and this hydrogen gas needs to be vented from the interior chamber of the trailer housing 10 to prevent various obvious dangers.

One embodiment supplements the mobile labs with Central Lab Processing. The collection units or CUs leverage the lab processing of each Deployment to collect samples at remote locations, bringing the testing to specific locations or groups of employees and/or guests. Leveraging CUs or collections at the various deployments, processing could also be shipped to the Central Processing Lab. Once tests are received there is a 24-48 hours processing to results time. On-Location Point(s) of Contact (POC) will be provided to receive and help coordinate efforts with our staff and to safety brief them and handle them from arrival to departure.

Testing and sample processing procedures built into the workflow include collection units ranging from 2-person teams, to pop-up tents, to trailer-based collection units and laboratories, and to truck-based mega-laboratories with throughputs of several thousand samples per day. The established workflows and procedures are not limited to just COVID testing but can be used to test for almost any virus and bacteria or be used to administer vaccinations as they become available. The laboratories are equipped with the best industry-standard instrumentation and automation available. They are designed to be easily retrofitted for blood or urine-based testing ranging from drug testing to cancer screening. The unique design and approach ensure that the customer needs are met at multiple levels. The embodiment provides an integrated solution: administration, sampling, testing, results communication, billing, reimbursement, and secure digital storage for test results—for an office, employees, and guests. FDA certified testing is done using the mobile lab described above, and cloud-based app and mobile app are provided for employees to manage their own test results, vaccination records and health certifications. This is done securely and enables sharing with their families and employers. The employer has access to an enterprise application which can be used by to manage, analyze, and track tests results for employees supporting corporate safety and testing policies. The testing lab can be SAFETY Act Certified—As certified by the Department of Homeland Security. The labs can also conform to requirements of the Clinical Laboratory Improvement Amendments (CLIA). The Centers for Medicare & Medicaid Services (CMS) regulates all laboratory testing (except research) performed on humans in the U.S. through the Clinical Laboratory Improvement Amendments (CLIA). In total, CLIA covers approximately 260,000 laboratory entities. The Division of Clinical Laboratory Improvement & Quality, within the Quality, Safety & Oversight Group, under the Center for Clinical Standards and Quality (CCSQ) has the responsibility for implementing the CLIA Program. The objective of the CLIA program is to ensure quality laboratory testing. Although all clinical laboratories must be properly certified to receive Medicare or Medicaid payments, CLIA has no direct Medicare or Medicaid program responsibilities.

One exemplary flow for an employer set up is as follows:
1. Approval to Proceed—Completion of contract signing.
2. Site Survey—Site-specific survey to include location of sample collection station, power, water, bathroom, flow of people to be tested while maintaining social distancing, and safety plan including point of contact in case of emergency.
3. Personnel—The following personnel will be provided to carry out all aspects of the process:
I. Ordering Physician to order the tests, answer questions, and provide any necessary follow up once test results are received.
II. Sample Collection Technicians that are trained in carrying out the collection process
III. Laboratory Technicians to process and record all samples IV. Site coordinator to act as primary point of contact and to ensure protocols and procedures are executed according to the contract
4. Delivery—All equipment, supplies, and support materials to ensure effective and safe sample collection and processing will be delivered and set up per the executed contract agreement.
5. Testing Notification and Communication—An email notification will be provided to employer/customer detailing the sample collection procedure with links to process videos, documentation, FAQ's and on-line sign up procedures. The notification is intended to inform all persons on the process, enter the necessary information, and to schedule an appointment.
6. Training A half-day of set up and training will be provided for all persons that will be directly involved in sample collection and processing.
7. Consumables All necessary test kits, PPE, sanitizing agents, and supplies will be delivered, inventoried, and stocked during the set up and training phase.
8. Sample Collection—Samples will be taken per the established protocol as quickly as possible to reduce que wait times. Via auto drive through, or CU.
9. Sample Processing—Samples will be received in the laboratory and processed using a lab with CLIA Certified test protocol.
10. Test Results—Upon completion of sample processing, test results will be recorded, and a summary of all results sent to the customer/employer. Individual results will be sent to each person that received testing.
11. Take Down—When sample collection is complete, all equipment and supplies will be removed from customer premises and the premises returned to the same condition as found.
12. Waste—All hazardous waste will be removed and disposed of according to county regulations. Non-hazardous waste will be disposed of by customer.

In one embodiment for the hospitality application, the following features are available:

1. Server App can be segmented into multiple data sets:

a. Location, Venue, Event

b. Categories: Employees, Guests, Other

c. Sub-Categories: Teams, user-defined sub-categories

2. Mobile App (for Employees, Guests) has an embedded verified Test Result credential (QR Code) which can authenticated by a Scan module (IOS, Android) and/or the Scan API (for CLEAR or other devices)

3. The app can be integrated into the existing hospitality IOS/Android Mobile Apps for the authenticated, verified Test Result QR Code. LifeSite Plus can also be a co-branded, separate application also. Mobile and Internet.

4. The authenticated, verified Test Result can be integrated into the enterprise guest check-in application(s)

5. Employees and Guests can be loaded into the server app from the existing HR System(s) and guest systems. This can be persistent storage or storage by dates, event, etc.

6. The authenticated, verified Test Result can be integrated into the enterprise Time & Attendance system for employees

7. Server app would have an aggregate view to manage, track and support tracing of test results

a. Test Results would be reported in real-time to the server/mobile app

b. The app helps manage the consent process to ensure HIPAA compliance and data protection

c. Additional lab results can also be accommodated through the additional integrations directly to additional labs or through a manual process (though marked as “Manually Verified”)

8. The entire solution can be integrated with a “wellness, monitoring solution” ie. temperature checking/scanning/monitoring, wellness questionnaire, etc.

Policies and testing rules around event testing and “safety bubbles” can be established by event organizers and deployed in system rules.

While particular embodiments of this invention have been shown in the drawings and described above, it will be apparent that many changes may be made in the form, arrangement and positioning of the various elements of the combination. In consideration thereof, it should be understood that preferred embodiments of this invention disclosed herein are intended to be illustrative only and not intended to limit the scope of the invention.

What is claimed is:

1. A method to test a person, comprising
capturing genetic virus or bacteria material from a person being tested;
performing a RNA and or DNA or genetic test on the captured material for a predetermined pathogen;
if the test shows an absence of the predetermined pathogen, certifying the person with a pathogen free status; and
providing a certificate during travel or entrance to a facility as proof of pathogen free status.

2. The method of claim 1, comprising testing for COVID viruses.

3. The method of claim 1, comprising testing for SARS viruses.

4. The method of claim 1, wherein the certificate comprises a paper certificate, a digital certificate, a symbol, or a bar code.

5. The method of claim 1, comprising:
confirming an identity of person using biometrics from one or more of: an eye, a face, and a fingerprint;
using a camera, continuously check the confirmed person takes one or more personal samples and inserts the personal samples into a RNA and or DNA tester;
if the RNA or DNA tester indicates an absence of a pathogen, certifying the person with a first indication in the certificate and otherwise certifying the person with a second indication.

6. The method of claim 5, wherein the first indication comprises “Healthy” and the second indication comprises a test failed indication.

7. The method of claim 1, comprising
confirming an identity of person using biometrics from one or more of: an eye, a face, and a fingerprint;
using a camera, continuously check the confirmed person takes one or more personal samples and with a chain of custody providing the personal samples into a DNA tester;
if the RNA or DNA tester indicates an absence of a pathogen, certifying the person with a first indication in the certificate and otherwise certifying the person with a second indication.

8. The method of claim 1, wherein the chain of custody secures delivery of the personal samples to a mobile testing lab or a remote testing lab.

9. The method of claim 1, comprising scanning test result and providing access to a facility based on the test result.

10. The method of claim 1, comprising spraying a disinfectant using an electrostatic sprayer.

11. The method of claim 1, comprising providing a learning system or a neural network that determines likelihood that person has been exposed to COVID virus.

12. The method of claim 11, comprising determining if the person is unlikely to be COVID positive, instructing a testing lab to pooled test samples to speed up test capacity, and otherwise individually testing the person's specimen.

13. The method of claim 1, comprising performing COVID tests with a mobile testing lab.

14. The method of claim 13, wherein the mobile testing lab comprises:
a mobile test platform including a sealed environment that is easily disinfected;
a plurality of storage spaces to store virus genetic testing equipment, diagnostic computer, support equipment, a fume hood with an internal filter and no outside venting; and
a power generator.

14. The method of claim 13, wherein the mobile testing lab further comprises:
a self-contained bio safety cabinet, no outside venting with HEPA filtration;
a PCR Unit positioned therein; and
a centrifuge to receive tubes.

15. The method of claim 13, wherein the mobile testing lab further comprises: RNase-Free pipet tips with aerosol barrier for beta mercaptoethanol (beta-ME); pipettes are be packaged to accommodate isolations; one or more mixers with heads; a heating block; a thermometer; a thermomixer; an Ice Bucket; disposable gloves; a biohazard waste bin and bag; bench coat paper; PCR Tubes; tube strips; and tube holders.

16. The method of claim 1, wherein the digital certificate comprises a mobile app, an icon, a 2D bar code, a 3D bar code, or an entry on a blockchain.

17. The method of claim 1, wherein the digital certificate is optically or wirelessly scanned at an access station and based on the reports contained therein, enabling a person to have clearance for travel, work, or study.

18. The method of claim 18, comprising applying micro-positioning data from ultrawideband radios and contact tracing people exposed to the pathogen.

19. A method to test a person, comprising
checking an identity of a person using biometric data including eye or fingerprint biometrics;
authenticating the person and capturing genetic material from a person being tested while the person is authenticated to avoid fraud;
performing a RNA or DNA or genetic test on the captured material for a predetermined pathogen;
if the test shows an absence of the predetermined pathogen, certifying the person with a pathogen free status; and
providing a certificate during travel or entrance to a facility as proof of pathogen free status.

20. The method of claim 19, comprising using a camera to capture user biometrics and continuity of user test sampling, shipping, and testing to reduce test tampering and applying micro-positioning data from ultrawideband radios and contact tracing people exposed to the pathogen.
